(19) United States
(12) Patent Application Publication
(10) Pub. No.: US 2023/0190947 A1
(43) Pub. Date: Jun. 22, 2023

(54) Hemoglobin-Targeted Drug Delivery For The Treatment of Cancer

(71) Applicant: Therapure Biopharma Inc., Mississauga (CA)

(72) Inventors: Steven Brookes, Mississauga (CA); J. Gordon Adamson, Mississauga (CA); David Bell, Mississauga (CA)

(21) Appl. No.: 16/310,197

(22) PCT Filed: Jun. 21, 2017
(86) PCT No.: PCT/IB2017/053719

§ 371 Date: Dec. 14, 2018

Related U.S. Application Data
(60) Provisional application No. 62/352,642, filed on Jun. 21, 2016.

(51) Int. Cl. A61K 47/64; A61P 35/04; A61P 35/00; A61P 1/00

(52) U.S. Cl. CPC A61K 47/6445; A61P 35/04; A61P 35/00; A61P 1/00

(57) ABSTRACT

The use of hemoglobin as a targeting carrier for drugs is disclosed. Such hemoglobin-drug complexes have utility in the treatment of cancer, in particular cancers of the liver and colon. Said drug is preferably a nucleoside analog, in particular floxuridine and is covalently attached to the hemoglobin in a molar drug ratio of between 1 and 20.

### RELATED APPLICATION

This application is a national phase entry application of Patent Cooperation Treaty Application No. PCT/IB2017/053719, filed Jun. 21, 2017 (which designates the U.S.), which claims the benefit under 35 USC § 119(e) from U.S. Provisional Application No. 62/352,642, filed on Jun. 21, 2016 (now abandoned), which are incorporated herein by reference in their entirety.

The present invention relates to a hemoglobin-floxuridine conjugate (Hb-FUdR) and its use in the treatment of cancer. More particularly, the anti-tumor efficacy of Hb-FUdR in a model of human colon cancer is provided.

The predominant uptake of Hb-FUdR is expected to be in the liver given the liver's capacity to take up hemoglobin. Surprisingly, the inhibition of tumour growth in the colon was observed, as well as an increase in overall survival. This was surprising given that the tumours were derived from the colon and the inhibition of the tumour was observed within the colon. This technology thus also appears to be applicable to non-liver tumors.

The invention provides, in one aspect, a method for the treatment of non-liver tumors by hemoglobin-drug complexes or hemoglobin mimics by incorporation of the complex into cells within tumors that are capable of interacting with and taking up hemoglobin. The invention extends to tumor associated macrophages (TAMs) as well as certain non-liver tumor cells that also express the CD163 receptor. The invention additionally extends to antibodies or other ligands to CD163.

In particular, the invention provides a method for treating liver and non-liver tumors comprising contacting the tumor with a hemoglobin-drug complex or hemoglobin mimic to inhibit growth of or shrink tumors and improve survival of the host.

In another embodiment, a method is provided for the treatment of non-liver tumors comprising contacting the tumor with a hemoglobin-drug complex or hemoglobin mimic to cause incorporation of the complex or mimic into cells within tumors bearing receptors for hemoglobin or via other mechanisms of uptake, and thereby affect growth of the tumor and survival of the host. Generally, this includes slowing the rate of growth of the tumor or halting growth of the tumor.

A method is also provided for treating liver and non-liver tumors comprising contacting the tumor with a hemoglobin-drug complex or hemoglobin mimic to effect incorporation into cells within tumors bearing receptors for hemoglobin.

In addition, a method is provided for the treatment colorectal cancer tumors comprising contacting the tumor with a hemoglobin-drug complex or hemoglobin mimic to cause incorporation of the complex or mimic into cells within tumors bearing receptors for hemoglobin or via other mechanisms of uptake.

In another embodiment, a method for the treatment colorectal cancer tumors is provided comprising contacting the tumor with a hemoglobin-drug complex or hemoglobin mimic to effect incorporation of the complex or mimic into cells within tumors bearing receptors for hemoglobin.

In another embodiment, there is provided the use of a therapeutic composition comprising a hemoglobin-drug complex or hemoglobin mimic for the treatment of cancer, for example liver and non-liver tumors or colorectal cancer tumors.

The drug may be a nucleoside analog, for example a nucleoside analog anticancer drug. In one embodiment, the hemoglobin-drug complex may be hemoglobin-floxuridine. The hemoglobin-floxuridine may have a molar drug ratio of between 1 and 20.

In a further embodiment, a hemoglobin mimic is employed. Typically, the hemoglobin mimic may be for example an antibody or antibody fragment or a peptide.

The hemoglobin is generally >99% pure hemoglobin sub-type A0. Moreover, the hemoglobin, or hemoglobin-drug complex or the hemoglobin mimic is capable of binding to CD163.

It is believed that the above observed effect arises because TAMs express receptors for hemoglobin such as CD163 which provide a mechanism by which Hb-FUdR may be localized to tumors by such hemoglobin-binding cells, not just in the liver but elsewhere in the body, since TAMs are tightly associated constituents of many tumours, in the liver and elsewhere. The data in the colorectal cancer model discussed below support this conclusion. TAMs expressing CD163 are reported in the literature.

The drug delivery technology discussed herein is based upon the attachment of therapeutic drugs to hemoglobin (Hb) for targeted delivery to the liver. This takes advantage of the body's natural mechanism of clearing cell-free Hb through the liver. This is a high capacity system capable of processing about 6 grams of Hb daily, offering the potential for Hb to serve as an effective carrier of drugs to the liver. Through an active process of Hb uptake, attached drugs are effectively delivered to cells of the liver. The present invention serves to provide drug candidates for the treatment of liver cancer (Hb-FUdR) and other tumors. Many liver cancer patients remain poorly served by current liver cancer therapies. There is a need for targeted chemotherapeutic drugs that demonstrate reduced side effects.

Hb-FUdR is a floxuridine-hemoglobin drug conjugate (FUdR-HDC). Floxuridine (FUdR) is a cytotoxic nucleoside analogue fluoropyrimidine with a narrow margin of safety. For this reason, it is currently only used locoregionally for treatment of cancers of the liver via hepatic arterial infusion (HAI) in an effort to minimize systemic toxicities. Hb-FUdR offers the potential for increased efficacy and safety through improved targeting of FUdR to the liver following intravenous infusion without the need for complicated locoregional administration. Hb-FUdR may also be efficacious in treating metastatic tumors in the liver such as those that arise from late-stage colorectal cancer, either via a sinusoidal bystander effect, or by specifically targeting tumor-associated macrophages (TAMs), which have the capacity to take up HDCs via cell surface receptors for Hb and release the attached drug to act locally against the tightly associated tumor cells.

Primary liver cancer, or hepatocellular carcinoma (HCC), is the second most common cause of death from cancer worldwide. There are over 700,000 new cases of liver cancer each year and nearly as many deaths. The prevalence is growing due to increasing rates of hepatitis C virus (HCV) infection—the primary cause of liver cancer (WHO 2008 estimates, GLOBOCAN). The most effective treatment of HCC in North America is liver transplant or surgical resection. However, only a small percent (10-20%) of liver cancer patients qualify for surgery (early stage or localized HCC) since diagnosis of the disease is often too late. Intermediate stage liver cancer patients who do not qualify for surgery are presented with few treatment options, none of which are curative and which only provide a modest increase in overall survival: these include locoregional modalities such as trans-arterial chemoembolization (TACE), and radiofrequency ablation among others. There is no widely accepted standard of care for the treatment of liver cancer, chemotherapy or otherwise.

Chemotherapy (i.v. infusion or oral) has an historical response rate in HCC of only ˜20%. It is typically only offered in advanced stages of the disease when other methods are not an option or have failed. Sorafenib (Nexavar®), a drug that has been adopted by many as a standard of care since late 2007, extends survival by only three months (10.7 months overall survival vs. 7.9 months) with no improvement in quality of life (SHARP trial). Furthermore, toxicity from sorafenib therapy is often dose-limiting leading to cessation of therapy. Newer, better and less toxic chemotherapeutic agents are clearly needed for liver cancer treatment.

Standard chemotherapy drugs such as doxorubicin and cisplatin are administered as “cocktails”, either in systemic intravenous regimens when appropriate or, more commonly, via TACE, for patients diagnosed with intermediate stage HCC. Fluoropyrimidines such as 5-fluorouracil (5-FU) and FUdR, a more potent version of the more commonly used 5-FU, have also been evaluated systemically but are less effective due to dose-limiting toxicities. FUdR is considered too toxic for systemic i.v. administration and is no longer used in standard intravenous chemotherapy for any cancer. However, FUdR exhibits high liver uptake and tumor shrinkage when administered via HAI, demonstrating better uptake and response than systemic 5-FU for patients with colorectal cancer liver metastasis (CRCLM), albeit with considerable dose-limiting liver toxicities that are associated with the level of drug required for efficacy. There are also significant technical challenges and safety concerns around HAI administration itself; for example, pump complications. As such, use of HAI FUdR therapy for liver cancer patients (both HCC and CRCLM) has been limited primarily to select treatment centers. For reasons described in the sections below, the compound FUdR-HDC (Hb-FUdR) of the invention is designed to address the limitations associated with effective use of FUdR (and fluoropyrimidines in general) for the treatment of liver cancer. Because of the inherent liver-targeting properties of Hb, Hb-FUdR can be considered a safer, easier-to-administer alternative to current locoregional therapies (HAI FUdR, TACE), with the potential to treat both primary HCC as well as CRCLM.

Hb-FUdR is a synthetic conjugate of FUdR and hemoglobin designed to deliver FUdR to the liver by taking advantage of the well-documented, natural clearance pathways for hemoglobin, predominantly by the liver.

In support of hemoglobin-drug complexes targeting the liver and key cells involved in infection and inflammation such as macrophages, the inventors have shown an improvement in anti-viral response and overall health in mice infected with a lethal hepatitis virus known to cause liver failure using a liver-targeted hemoglobin-antiviral conjugate (hemoglobin-ribavirin conjugate) analogous to Hb-FUdR in terms of drug payload and releasable drug attachment chemistry (Brookes et al., 2006, Bioconjugate Chem 17, 530-7). A three-fold lower dose of the Hb-conjugated vs. free drug improved antiviral responses in vivo and even more markedly in vitro in virus-infected macrophages and hepatocytes (Levy et al., 2006, Hepatology 43, 581-91). This finding supports the concept of improved efficacy and potency via liver and macrophage targeting using Hb as a carrier.

When conjugated to Hb, FUdR can be delivered to cells within the liver directly via systemic administration. Drug concentration in the liver can be increased upon multiple circulatory passes through the liver to expose primary HCC tumors or colorectal cancer liver metastases to higher drug concentrations than what can be safely achieved with systemic free drug delivery. Effective FUdR concentrations with Hb-FUdR can be achieved at relatively low Hb dose levels: For example, a dose of Hb known to be well tolerated in humans (<1.5 g) would provide a >100 μM liver concentration of FUdR shortly after injection/infusion. If this dose were given weekly, the drug levels achieved on a mg/kg basis are comparable to what is currently achieved with continuous HAI FUdR therapy. However, direct hepatic infusion using pumps is not needed for Hb-FUdR since IV infusion would accomplish what HAI can do without pumps, i.e., provide the drug to tumors (which predominantly feed from the hepatic artery) while minimizing systemic toxicity because of the liver-targeting nature of Hb. As with the free drug, additional tumor selectivity of FUdR-HDC within the liver would rely upon the metabolic and proliferative differences between healthy non-proliferating hepatocytes and macrophages, compared to rapidly proliferating cancer cells of the growing tumors.

Hb-FUdR shows enhanced in vitro cytotoxic effect against liver cancer cells and efficacy in vivo in mouse models of HCC and CRCLM. In a murine model of HCC Hb-FUdR prevented tumor growth in 7 out of 10 mice implanted with HepG2-derived human liver cancer tumors relative to control mice (2/10). In a murine model of CRC Hb-FUdR inhibited tumor growth and increased overall survival comparable to FUdR in mice implanted with human colon tumors in their cecum, demonstrating Hb-FUdR can be effective in tumors not located in the liver.

Hb-FUdR is a synthetic conjugate of floxuridine (FUdR) and hemoglobin (Hb) that binds endogenous haptoglobin (Hp) designed to deliver FUdR to cells expressing the receptor for the hemoglobin-haptoglobin (Hb-Hp) complex upon peripheral i.v. infusion. A Hb-Hp receptor, CD163, is present on the surface of macrophages including hepatic macrophages (Kupffer cells)¹. In addition, Hb and Hb-Hp have been shown to bind to hepatocytes in a receptor-mediated fashion, although the hepatocyte receptor has not yet been identified². Release of FUdR will occur upon endocytosis and degradation of the Hb carrier. Drug can therefore be delivered to cells within the liver directly and drug concentration in the liver can be increased upon multiple passes through the liver to expose local target tissue within the liver, such as primary HCC or colorectal metastases, to higher drug concentrations than achievable with systemic free drug delivery. Hb is taken up not only by hepatocytes but also by liver macrophages³, thereby making possible even higher local drug concentrations. Furthermore, the Hb-drug conjugation approach will target macrophages that are recruited by and infiltrate liver tumors, and the modulation of macrophage cytokines (as demonstrated for the Hb-ribavirin conjugate in Brookes et al. as a result of binding the Hb) may beneficially alter the pathological symbiotic relationship between the tumor and its associated macrophages. Furthermore it is known that the binding of Hb to CD163 has its own inherent anti-inflammatory effects which may be tumor modulating. ¹ Kristiansen, M., Graversen, J. H., Jacobsen, C., Sonne, O., Hoffman, H. J., Law, S. K., Moestrup, S. K. 2001 Identification of the haemoglobin scavenger receptor. Nature 409 (6817), 198-201² Okuda, M., Tokunaga, R., Taketani, S. 1992 Expression of haptoglobin receptors in human hepatoma cells. Biochim. Biophys. Acta 1136 (2), 143-9³ Kristiansen et al. 2001

Selective efficacy of Hb-FUdR for tumors in the liver would in part be a function of the metabolic differences between healthy non-proliferating hepatocytes and macrophages and the rapidly proliferating cancer cells that have elevated expression of drug transporters (UT, hENT1), TP, TK and/or TS, i.e., selective efficacy would in part be a function of the inherent nature of FUdR itself as it was originally designed, which should spare healthy hepatocytes and macrophages in the hepatic circulation (not counting any direct delivery of HDC via purported Hb-Hp receptors, which may also be over-expressed relative to hepatocytes and macrophages).

In addition to the potential for delivery of FUdR directly to HCC cells via Hb uptake, there is a second mechanism available for delivery of FUdR into the HCC cells. This mechanism (illustrated above in FIG. 5) involves indirect delivery of the drug via the tumor infiltrating macrophages (also called tumor associated macrophages or TAMs) of the liver tumors⁴. This method of delivery would make use of the Hb-Hp receptors such as CD163 which expressed on the surface of macrophages, including TAMs^{5,6,7}. Following receptor-mediated uptake of the drug conjugate, free FUdR will be released within the macrophages. Studies have demonstrated the ability of macrophages to release FUdR and 5-FU when taken up in liposomes^{8, 9}. This method of macrophage-mediated delivery of chemotherapy has been also demonstrated in vivo for other compounds such as doxorubicin¹⁰. Therefore, drug delivery to the macrophages will provide a means for delivery of FUdR and/or 5-FU to tightly associated HCC cells, possibly in a sufficiently high enough local concentration to overcome the inactivation/removal pathways found within the HCC cells. By this localization mechanism, selective efficacy would rely upon the metabolic difference between healthy non-proliferating hepatocytes and macrophages and the rapidly proliferating cancer cells at the periphery of the growing tumors that have elevated expression of TK and/or TS, i.e., efficacy would be a function of the inherent nature of the drug itself as it was originally designed. ⁴ Peng, S. H., Deng, H., Yang, J. F., Xie, P. P., Li, C., Li, H., Feng, D. Y. 2005 Significance and relationship between infiltrating inflammatory cell and tumor angiogenesis in hepatocellular carcinoma tissues. World J. Gastroenterol. 11(41), 6521-24⁵ Takai, H., Kato, A., Kato, C., Watanabe, T., Matsubara, K., Suzuki, M., Kataoka, H. 2009; The expression profile of glypican-3 and its relation to macrophage population in human hepatocellular carcinoma. Liv. Int 7, 1056-64⁶ Kawamura, K., Komohara, Y., Takaishi, K., Katabuchi, H., Takeya, M. 2009 Detection of M2 macrophages and colony-stimulating factor 1 expression in serous and mucinous ovarian epithelial tumors. Pathol. Int 59(5), 300-05⁷ Sica, A., Schioppa, T., Mantovani, A., Allavena, P. 2006 Tumour-associated macrophages are a distinct M2 polarised population promoting tumour progression: potential targets of anti-cancer therapy. Eur. J. Cancer 42, 717-27⁸ Ikehara, Y., Niwa, T., Biao, L., Ikehara, S. K., Ohashi, N., Kobayashi, T., Shimizu, Y., Kojima, N., Nakanishi, H. 2006 A carbohydrate recognition-based drug delivery and controlled release system using intraperitoneal macrophages as a cellular vehicle. Cancer Res., 66(17), 8740-48⁹ van Borssum Waalkes, M., Kuipers, F., Havinga, R., Scherphof, G. L. 1993 Conversion of liposomal 5-fluoro-2′-deoxyuridine and its dipalmitoyl derivative to bile acid conjugates of alpha-fluoro-beta-alanine and their excretion into rat bile. Biochim. Biophys. Acta Mol. Cell Res. 1176(1-2), 43-50¹⁰ Storm, G., Steerenberg, P. A., Emmen, F., van Borssum Waalkes, M., Crommelin, D. J. 1988 Release of doxorubicin from peritoneal macrophages exposed in vivo to doxorubicin-containing liposomes. Biochim. Biophys. Acta. 965, 136-45

Recently it has been reported that primary rectal cancer cells of some rectal cancer patients express CD163^{[4]}. Although Hb-FUdR is intended for liver targeting and uptake, it is unknown whether Hb-FUdR might affect primary rectal cancer cells with the CD163 receptor, or whether tumour-infiltrating macrophages (TAMs) and Kupffer cells, which express CD163^{[5],[6]}, can be targeted for drug delivery and localization. In these ways, the CRC targeting principle would rely upon the targeting of Hp-Hb-FUdR to CD163-expressing cells, including macrophages, where the pre-prodrug would be converted into FUdR and/or 5-FU and released locally to interact directly with, or with tightly associated/intermingled, CRC cells in the tumor. ^{[4]} Shabo, I., Olsson, H., Sun, X. F., Svanvik, J. 2009 Expression of the macrophage antigen CD163 in rectal cancer cells is associated with early local recurrence and reduced survival time. Int. J. Cancer 125, 1826-31^{[5]} Nagorsen, D., Voigt, S., Berg, E., Stein, H., Thiel, E., Loddenkemper, C. 2007 Tumor-infiltrating macrophages and dendritic cells in human colorectal cancer: relation to local regulatory T cells, systemic T-cell response against tumor-associated antigens and survival. J. Transl. Med. 5, 62^{[6]} Atsushi, H., Norio, H., Sk. Md. Fazle, A., Kojiro, M., Takami, M., Morikazu, O. 2005 Expression of CD163 in the liver of patients with viral hepatitis Path. Res. Pract. 30, 379-84

### Materials and Methods

### EXAMPLE 1. HEMOGLOBIN-FLOXURIDINE CONJUGATE

Hb-FUdR is a synthetic conjugate of purified human hemoglobin (Hb) and the fluoropyrimidine anticancer drug floxuridine (FUdR), in which FUdR is covalently attached to Hb. The purified Hb used in the production of Hb-FUdR consists of >99% HbA₀, the predominant human Hb phenotype. Hb was extensively purified to >99% purity using a process described in U.S. Pat. No. 5,439,591 (1995).

### Synthesis of FdUMP-Im

Hb-FUdR was synthesized according to Scheme 1 and as described below.

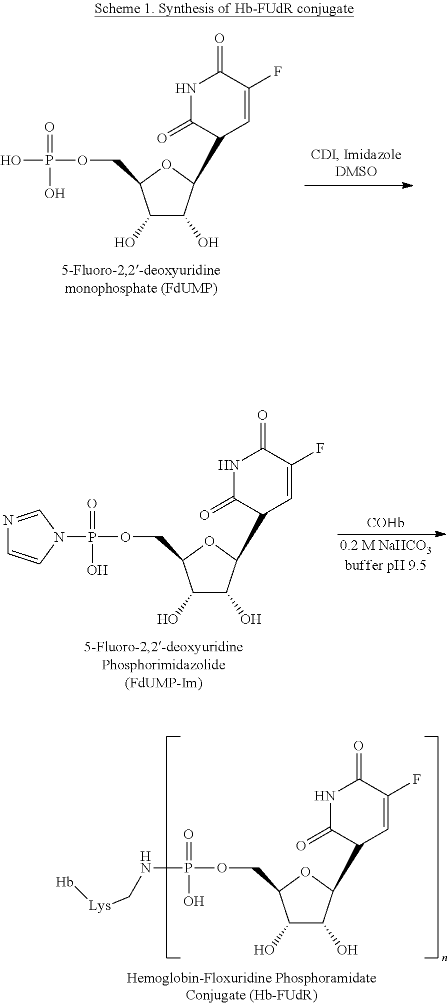

5-fluoro-2′-deoxyuridine-5′-monophosphate sodium salt (FdUMP, Sigma-Aldrich, 6.3 mg, 20 μmop was stirred in 1.0 mL of dimethyl sulfoxide DMSO in a pre-dried vial under a stream of dry N₂. In a separate pre-dried vial under dry N₂, 40 mg of carbonyl diimidazolide (CDI) was dissolved in 1.0 mL of DMSO. In a separate, third pre-dried vial under dry N₂, 30 mg of imidazole (Im) was dissolved in 2.0 mL of DMSO. To the stirred FdUMP suspension was added 470 μL of the CDI solution (116 μmol, ie. a 5.8-fold excess) and 500 μL of the Im solution (110 μmol, i.e. a 5.5-fold excess) under dry N₂. The suspension was rapidly stirred at RT. The final concentration of FdUMP was 3.2 mg/mL in DMSO. An additional 10 mg each of CDI and Im were dissolved in the reaction mixtures, which were then re-charged with N₂ and stirred at RT overnight. The CDI and Im additions (10 mg each) were repeated at 20, 22, and 26 h to complete the reaction by 27 h, as indicated by HPLC.

Monitoring by HPLC: 20 μL of reaction mixture was mixed with 180 μL of 200 mM NaHCO₃/Na₂CO₃ pH 9.5 buffer to quench and dissolve prior to injection (50 μL injection volume) onto an analytical C18 RP Aqua-Luna Phenomenex column, 66 mM K₂HPO₄ elution buffer pH 7.35, isocratic, 1 mL/min flow rate, absorbance monitored at 210, 280, and 254 nm.

The reaction mixture was transferred to a test tube and the reaction was stopped by freezing in liquid N₂. The frozen sample was lyophilized to remove DMSO. The lyophilized sample (a waxy, yellow solid) was sealed under N₂ and frozen at −20° C. until work-up and reaction with Hb

### Work-Up of FdUMP-Im

The waxy residue of the reaction mixture was dissolved in 500 μL of anhydrous ethanol (EtOH) to quench the excess CDI. To the anhydrous EtOH supernatant was added 1 mL of anhydrous ether (Et₂O) to cause precipitation/crystallization. The tube was put at −20° C. for 2 h to complete precipitation. The product was a fluffy white precipitate. This was centrifuged down to a pellet (5 min) and the supernatant was removed. The product was washed with 1 mL of Et₂O, centrifuged, and the Et₂O removed. This process was repeated 3 times. The final off-white powder product was dried under a gentle stream of dry N₂ in the tube and the final weight was determined (4.6 mg, 12.3 μmol, 63% yield).

Approximately ⅔^{rd} of the product was dissolved immediately in 300 μL of 200 mM NaHCO₃/Na₂CO₃ pH 9.5 buffer in preparation for reaction with COHb.

The other ⅓rd of the product was kept in a dried vial, charged with N₂, at −20° C., for supplemental addition to the conjugation reaction mixture at the 48 h mark.

### Conjugation of FdUMP-Im to COHb

In an eppendorf tube was placed 70 μL of 10 g/dL carbonmonoxyhemoglobin (COHb) in WFI (7 mg, 108 nmol). To this was added the 300 μL of FdUMP-Im solution followed by an additional 200 mL of 200 mM NaHCO₃/Na₂CO₃ pH 9.5 buffer. The reaction mixture was vortexed and the pH was monitored to maintain above pH 9.3 with 200 mM Na₂CO₃ pH >11 solution. The pH meter electrode was rinsed with 400 μL of 200 mM NaHCO₃/Na₂CO₃ pH 9.5 buffer (total reaction volume 970 μL). This was CO charged as best as possible, sealed with parafilm, and placed in a 37° C. water bath. After 3 h the pH was still 9.3. After 49 h of reaction, the remaining ⅓^{rd} portion of the FdUMP-Im solution was added to the reaction solution.

Progress of the reaction was monitored periodically over the course of 1 week by Anion Exchange HPLC using a Poros HQ 10 cm column eluted with a pH gradient (achieved using Tris and bis-Tris buffers outlined in Chart 1), a 4 mL/min flow rate, and a 504 injection volume.

CHART 1
Anion Exchange Chromatography
Elution Conditions

| | % | % | Duration to |
|---|---|---|---|
| | Solvent | Solvent | achieve next |
| Time (min) | A | B | gradient (min) |
| 0 | 100 | 0 | 1 |
| 1 | 0 | 100 | 7 |
| 8 | 0 | 100 | 2 |
| 10 | 100 | 0 | 2 |
| 14 | stop data | stop data | — |

Solvent A: 25 mM Tris pH 8.3 buffer
Solvent B: 25 mM bis-Tris pH 6.3 buffer

After one week (190 h) the reaction was considered complete with <10% unreacted Hb remaining; the reaction mixture was CO charged and frozen at −80° C. until further characterization.

Characterization by methods described in Brookes et al. 2006 confirmed the test article Hb-FUdR to have a molar drug ratio (MDR) of 4-8.

### Human Colon Cancer Model:

A study testing Hb-FUdR in mice orthotopically implanted with tumor fragments derived from colon cancer cells (transfected with green fluorescence protein for in-life tumor visualization was conducted as follows:

Study design: Fifty (50) tumor-bearing mice were randomly divided into five groups with ten (10) mice per group. Animals (10 mice per group) were treated with Hb-FUdR at 6, 32, and 161 mg Hb/kg (0.15, 0.74, and 3.7 mg FUdR/kg) twice weekly via intravenous injection (tail vein) at 5 mL/kg. Control untreated animals were treated with PBS at 5 mL/kg. Control FUdR treated animals were treated with 3.7 mg FUdR/kg.
Study endpoint: The study was terminated 47 days post tumor inoculation. All remaining mice in each group were sacrificed at this time point. All animals in the PBS treated group and approximately 50% of animals in other treated groups died before the study endpoint. Therefore, survival time between groups was considered one of the criteria to assess the efficacy of the test agents. Primary tumors were excised and weighted at necropsy for subsequent analysis.

### Results:

The anti-tumor efficacy of Hb-FUdR against the implanted human colon cancer was evaluated by comparing the primary tumor sizes measured twenty-one days after treatment initiation, and the differences in survival time between the Hb-FUdR treated and PBS control groups.

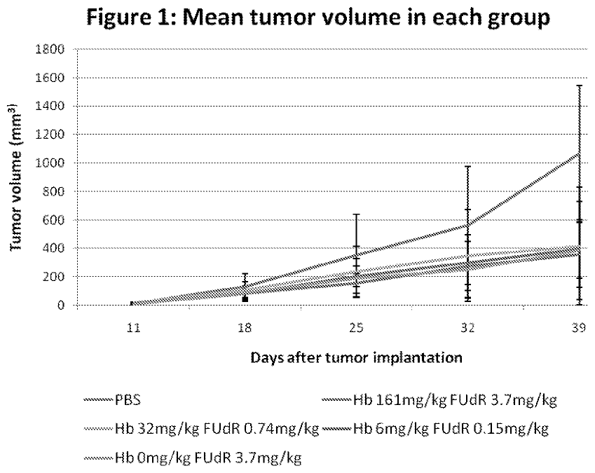

Efficacy of treatment on tumor size: Average tumor volume measured in each Hb-FUdR treated group and the corresponding p value comparison to the PBS treated group (Group 1) are shown in Table 2 below. There were no statistically-significant differences in the tumor volumes in any of the Hb-FUdR treated groups by comparison to the PBS treated group. But a trend of reduction in tumor size could still be seen in all Hb-FUdR treated groups especially in the high dose treated groups. Curves of the mean tumor volume in each group can be seen in FIG. 1.

TABLE 1
Efficacy on tumor volume

| | | Average tumor |
|---|---|---|
| Group | Agent | volume (mm³) |
| 1 | PBS | 563.63 ± 413.91 |
| 2 | Hb 161 mg/kg | 274.99 ± 221.77 |
| | FUdR 3.7 mg/kg | |
| 3 | Hb 32 mg/kg | 351.53 ± 323.25 |
| | FUdR 0.74 mg/kg | |
| 4 | Hb 6 mg/kg | 301.27 ± 195.4 |
| | FUdR 0.15 mg/kg | |
| 5 | Hb 0 mg/kg | 254.58 ± 196.74 |
| | FUdR 3.7 mg/kg | |

Efficacy of treatment on tumor metastasis: Tumor metastases were found in the liver and mesentery lymph nodes. The incidence of tumor metastasis to the liver and local lymph nodes in each Hb-FUdR treated group was compared to the PBS treated group. Table 3 below shows the total number of lymph node and liver metastases in each group in comparison to the PBS treated group. There were no statistically significant differences in lymph node or liver metastases between any of the Hb-FUdR treated groups and the PBS treated group.

TABLE 2
Comparison of tumor metastases in each group

| | | | No. of | p-value | No. of |
|---|---|---|---|---|---|
| | | No. of | mice | (Fisher's | mice |
| | | mice | with L.N. | exact | with liver |
| Group | Agents | evaluated | met. | test) | met. |
| 1 | PBS | 9 | 6 | — | 4 |
| 2 | Hb 161 mg/kg | 10 | 7 | 1.0 | 4 |
| | FUdR 3.7 mg/kg | | | | |
| 3 | Hb 32 mg/kg | 10 | 5 | 0.65 | 2 |
| | FUdR 0.74 mg/kg | | | | |
| 4 | Hb 6 mg/kg | 10 | 4 | 0.37 | 1 |
| | FUdR 0.15 mg/kg | | | | |
| 5 | Hb 0 mg/kg | 9 | 6 | 1.0 | 4 |
| | FUdR 3.7 mg/kg | | | | |

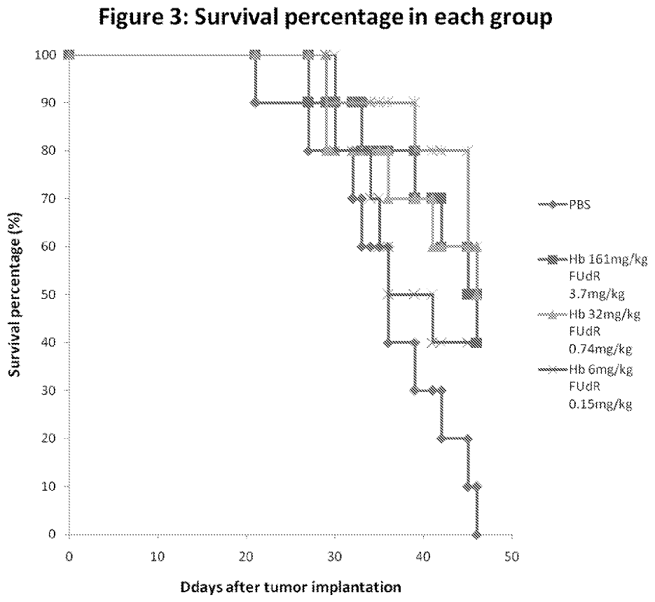

Efficacy of treatment on survival time: The difference in the survival between each Hb-FUdR treated group and the PBS treated group was compared. Table 3 below shows the mean survival time in each Hb-FUdR treated group in comparison to the PBS control (group 1). Statistically-significant differences can be seen in all of the Hb-FUdR treated groups, except the Hb 6 mg/kg-FUdR 0.15 mg/kg treated group (group 4), by comparison to the PBS control (The survival curves of each group can be seen in FIG. 3).

TABLE 3
Comparison of survival time in each group

| | | Mean survival | p-value |
|---|---|---|---|
| Group | Agents | time (days) | (log-rank test) |
| 1 | PBS | 35.7 | — |
| 2 | Hb 161 mg/kg | 42.4 | 0.018 |
| | FUdR 3.7 mg/kg | | |
| 3 | Hb 32 mg/kg | 44.1 | 0.014 |
| | FUdR 0.74 mg/kg | | |
| 4 | Hb 6 mg/kg | 39.4 | 0.118 |
| | FUdR 0.15 mg/kg | | |
| 5 | Hb 0 mg/kg | 43.9 | 0.003 |
| | FUdR 3.7 mg/kg | | |

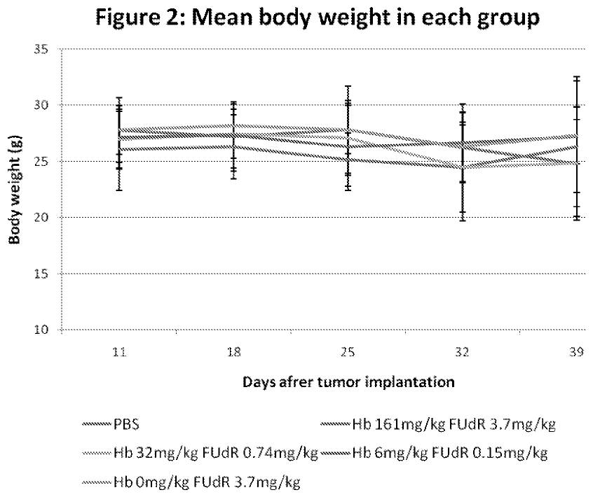

Estimation of compound toxicity: The mean body weight in all groups of mice was maintained within the normal range during the entire experimental period (Mean body weight in each group during the experimental period can be seen in FIG. 2). No weight losses were observed in any treated group during the experimental period. Other symptoms of related toxicity were absent by gross observation.

The data indicates that Hb-FUdR and FUdR significantly prolonged survival time of the treated mice without obvious toxicity.

### EXAMPLE 2

TBI 304, an antibody to CD163, was labelled using the Alexa Fluor 488 dye with 3.4 fluors per protein and binding to a cell line engineered to express recombinant human CD163 was demonstrated by flow cytometry.

1: A method for treating liver and non-liver tumors comprising contacting the tumor with a hemoglobin-drug complex, or mimic of a hemoglobin-drug complex, to thereby affect the growth of the tumor and survival of the host.

2: The method of claim 1 comprising contacting the tumor with the hemoglobin-drug complex or mimic to cause incorporation of the complex or mimic into cells within tumors bearing receptors for hemoglobin or via other mechanisms of uptake and thereby affect growth and survival of the tumor.

3: The method of claim 1 comprising contacting the tumor with a hemoglobin-drug complex or mimic to effect incorporation into cells within tumors bearing receptors for hemoglobin.

4: A method for the treatment colon cancer tumors comprising contacting the tumor with a hemoglobin-drug complex or mimic a hemoglobin-drug complex to cause incorporation of the complex or mimic into the tumor cells and/or cells within tumors bearing receptors for hemoglobin or via other mechanisms of uptake.

5: The method of claim 4 comprising contacting the tumor with the hemoglobin-drug complex or mimic to affect incorporation of the complex or mimic into cells within tumors bearing receptors for hemoglobin.

6: A method according to claim 1, wherein the drug is a nucleoside analog.

7: A method according to claim 1, wherein the drug is a nucleoside analog anticancer drug.

8: A method according to claim 1, wherein the hemoglobin-drug complex is hemoglobin-floxuridine.

9: A method according to claim 8 wherein the hemoglobin-floxuridine has a molar drug ratio of between 1 and 20.

10: A method according to claim 1, wherein the mimic of a hemoglobin-drug complex comprises a hemoglobin mimic.

11: A method according to claim 10 wherein hemoglobin mimic is an antibody or antibody fragment.

12: A method according to claim 1 wherein the conjugate is a hemoglobin-drug conjugate.

13: A method according to claim 12, wherein the hemoglobin is >99% pure hemoglobin sub-type A0.

14: A method according to claim 1, wherein the hemoglobin, hemoglobin-drug complex or hemoglobin mimic binds to CD163.

15: A method according to claim 4, wherein the drug is a nucleoside analog.

16: A method according to claim 4, wherein the drug is a nucleoside analog anticancer drug.

17: A method according to claim 4, wherein the hemoglobin-drug complex is hemoglobin-floxuridine.

18: A method according to claim 17, wherein the hemoglobin-floxuridine has a molar drug ratio of between 1 and 20.

19: A method according to claim 4, wherein the mimic of a hemoglobin-drug complex comprises a hemoglobin mimic.

20: A method according to claim 19, wherein hemoglobin mimic is an antibody or antibody fragment.
